(19) United States
(12) Patent Application Publication
(10) Pub. No.: US 2007/0048386 A1
(43) Pub. Date: Mar. 1, 2007

(54) MEDICAMENT FOR TREATING INFLAMMATORY AND NON-INFLAMMATORY ARTHRITIS

(76) Inventors: Ottavio Mallozzi SR., New Canaan, CT (US); Joan M. Fallon, Yonkers, NY (US)

Correspondence Address: MAINE & ASMUS, 100 MAIN STREET, P O BOX 3445, NASHUA, NH, 03061-3445, US

(21) Appl. No.: 11/468,384

(22) Filed: Aug. 30, 2006

Related U.S. Application Data
(60) Provisional application No. 60/712,552, filed on Aug. 30, 2005.

(51) Int. Cl. A61K 36/8945; A61K 36/28; A61K 35/12; A61K 31/60; A61K 31/7008

(52) U.S. Cl. 424/522; 424/769; 424/773; 424/764; 514/62; 514/159

(57) ABSTRACT

A medicament for the treatment of inflammatory and non-inflammatory arthritis that includes the following ingredients: arnica, calendula, glucosamine, salicylic acid, hamamelis, emu oil and wild yam. The medicament may be in the form of an ointment, cream, gel, emulsion or liquid solution that is topically applied directly to the affected area. Alternatively, a pad saturated with the medicament having self adhesive qualities that may be placed directly on the affected area may be used. Additionally, the medicament may be applied directly on the affected area or a pad saturated with the medicament is applied to the affected area, and then the area is wrapped with a sleeve or band.

### RELATED APPLICATIONS

This application claims the benefit of U.S. Provisional Application No. 60/712,552 filed Aug. 30, 2005. This application is herein incorporated in its entirety by reference.

### FIELD OF THE INVENTION

The present invention relates generally to an arthritis treatment and more specifically it relates to a medicament for treating inflammatory and non inflammatory arthritis and for providing pain relief for inflammatory and non inflammatory arthritis using natural, externally applied medications.

### BACKGROUND OF THE INVENTION

Arthritis is a disease characterized by inflammation of the joints, such as the knees, wrists, elbows, fingers, toes, hips, shoulders, neck and back. The inflammation is often accompanied by other symptoms, such as pain, swelling, stiffness, weakness, and deformity. The symptoms of arthritis may have a gradual or sudden onset and the pain associated with the symptoms may be sharp or a dull ache.

The most common types of arthritis are osteoarthritis, rheumatoid arthritis and fibromyalgia. Degenerative joint disease, or osteoarthritis, is the most common form of arthritis striking 121 people out of 1,000 between the ages of 18 and 79. According to the Arthritis Foundation, approximately 16 million Americans currently suffer from osteoarthritis. Three times as many women than men are osteoarthritis patients.

When bone rubs against bone because the joint surface is gone, osteoarthritis develops. This rubbing happens when the cartilage that surrounds the ends of the bone degenerates. Cartilage provides shock absorbency and reduces friction as a joint moves. A joint consists of two or more bones and the cartilage, which helps cushion the joint. Joints can come in many sizes and shapes. Most often the weight-bearing joints, such as the hips, knees and spine, are affected with osteoarthritis.

Rheumatoid arthritis is the most common form of inflammatory arthritis, affecting about two to three million Americans. It usually first appears between the ages of 25 and 50, but it may also occur in children and the elderly. Most commonly the joints of the fingers, wrists, arms and legs are affected and it will involve the same joints on both sides of the body. Swelling, pain, deformity and stiffness are typically present. Unlike osteoarthritis, rheumatoid arthritis may also affect the heart, lungs, and eyes of some patients. Rheumatoid arthritis can also cause an overall feeling of sickness and fatigue as well as weight loss and fever. Some patients with rheumatoid arthritis experience constant symptoms while others have courses of bad periods or flares and good periods called remissions. The cause of rheumatoid arthritis is unknown but it is thought to be an autoimmune disease meaning that the body tissue is the victim of an immune response against itself.

A common but misunderstood disease, fibromyalgia currently affects approximately five million Americans. Eighty to ninety percent of all patients are women between the ages of 35 and 60. Constant fatigue, deep muscle pain, sleeplessness and depression are widespread symptoms of fibromyalgia. Tender points under the skin have become a hallmark of this disease. For all patients, these tender points are painful when pressed.

Fibromyalgia syndrome (FMS) does not involve the joints like osteoarthritis and rheumatoid arthritis. Rather, it is a type of rheumatism of the muscles, ligaments, tendons or soft tissues whereas rheumatism describes the stiffness and pain associated with arthritis. Although the symptoms of fibromyalgia may come and go over the years, the disease itself is chronic or long-term.

It can be appreciated that a variety of arthritis treatments have been in use for years. Typically, arthritis treatments are comprised of non-steroidal anti-inflammatory drugs (NSAIDs), Cox-2 inhibitors, corticosteroids, other medicaments, such as glucosamine and chondroitin, alternative treatments, such as acupuncture and magnet therapy, and various types of surgeries, including arthroscopic debridement and lavage.

The main problem with conventional arthritis treatments are that the non-steroidal anti-inflammatory drugs (NSAIDs) can only be taken for limited periods of time without major side effects, such as ulcers. This is true of other medications, such as corticosteroids, which when used in large doses over a period of time can cause weight gain, thinning of the skin, cataracts and osteoporosis. The Cox-2 inhibitors have been found to increase the amount of cholesterol and other fatty byproducts, thus increasing the risk of heart attack. Another problem with conventional arthritis treatments are that surgery, while potentially helpful in the immediate weeks and months following the surgery, has very little long term efficacy. Another problem with conventional arthritis treatments are that none of the interventions provide long term relief.

What is needed, therefore, is a medicament for treating inflammatory and non inflammatory arthritis that substantially departs from conventional concepts and prior designs, and in so doing provides a treatment primarily developed for the purpose of providing pain relief for inflammatory and non-inflammatory arthritis using a natural, externally applied medication.

### SUMMARY OF THE INVENTION

It is a goal of the present invention to provide a medicament for treating inflammatory and non inflammatory arthritis that will overcome the shortcomings of the conventional arthritis treatments.

One embodiment of the present invention provides a medicament for treating inflammatory and non inflammatory arthritis that is an ointment, cream, gel, emulsion or liquid solution applied directly to the affected area and contains natural ingredients to help with the pain of arthritis.

Another embodiment of the present invention provides a pad that contains a medicament for treating inflammatory and non inflammatory arthritis that can hold the solution mixture in the form of a pad or pads with self adhesive qualities that may be placed directly on the affected area.

In a further embodiment of the present invention, the medicament is applied directly on the affected area and the area is wrapped with a cotton, woolen or other material sleeve or band. Alternatively, a pad saturated with the medicament is applied to the affected area, then wrapped with a sleeve or band.

In one embodiment of the present invention, the formulation of the medicament includes the following: arnica, calendula, glucosamine, salicylic acid, hamamelis, emu oil and wild yam.

The features and advantages described herein are not all-inclusive and, in particular, many additional features and advantages will be apparent to one of ordinary skill in the art in view of the drawings, specification, and claims. Moreover, it should be noted that the language used in the specification has been principally selected for readability and instructional purposes, and not to limit the scope of the inventive subject matter.

### DESCRIPTION OF THE DRAWINGS

FIG. 1 is a graph demonstrating the changes in the pain scale of the individuals treated with the medicament having the composition described in Formula 1.

FIG. 2 is a graph demonstrating the changes in the activities of daily life of the individuals treated with the medicament having the composition described in Formula 1.

FIG. 3 is a graph demonstrating the changes in the pain scale of the individuals treated with the medicament having the composition described in Formula 2.

FIG. 4 is a graph demonstrating the changes in the activities of daily life of the individuals treated with the medicament having the composition described in Formula 2.

FIG. 5 is a graph demonstrating the changes in the pain scale of the individuals treated with the medicament having the composition described in Formula 3.

FIG. 6 is a graph demonstrating the changes in the activities of daily life of the individuals treated with the medicament having the composition described in Formula 3.

FIG. 7 illustrates a pad impregnated with the medicament of the present invention having an adhesive to facilitate adhering the pad to an affected area.

FIGS. 8A-C illustrate various examples of sleeves and bands that may be wrapped directly around an affected area or used to hold a pad containing the medicament in place on the affected area.

### DETAILED DESCRIPTION

In one embodiment, the active ingredients of the medicament include the following: arnica, calendula, glucosamine, salicylic acid, hamamelis, emu oil and wild yam. It should be noted that alternative ingredients may be used. For example, members of the Inula and Scorzonera families may be used in place of arnica or calendula. Chondroitin sulfate or methylsulfonylmethane (MSM) may be used in place of glucosamine. Members of the Parrotiopsis, Fothergilla and Sycopsis families may be used in place of hamamelis. Oil from other ratite birds, such as ostrichs, kiwis, cassowaries, and rheas, may be used in place of emu oil. Members of the Viburnum, Apium, Althaea, and Filipendula families may be used in place of wild yam.

Arnica (Arnica montana) is a perennial herb having discutient properties. Medicinally, a tincture made from the flower is used externally for the treatment of sprains, bruises and wounds. Calendula (Calendula officinalis) is a flower with pale green leaves and golden orange flowers. Medicinally, the flower is used as a remedy for the pain and swelling caused by insect stings when rubbed upon the affected area, while a lotion made from the flower is useful in treating sprains. Glucosamine is an amino sugar that builds and maintains cartilage, tendons, and other connective tissues in the body by acting as a building block for these materials and inhibiting enzymes that destroy cartilage. Salicylic acid is a crystalline organic carboxylic acid that functions as a plant hormone. The medicinal properties of salicylic acid include fever relief, mild antibiotic, and triggering a systemic immune response. Hamamelis (witch hazel) is a shrub with smooth grey bark. The medicinal properties of hamamelis are as an astringent and anti-inflammatory agent. Emu oil is an excellent trans-dermal carrier and is an effective anti-inflammatory. It is natural, non-toxic, non-comedogenic, and hypo-allergenic. Wild yam is a root plant that is used medicinally as an antispasmodic.

Various formulations of the medicament are illustrated below:

FORMULA 1

| | |
|---|---|
| 70-85% | petrolatum/petroleum jelly |
| 5-10% | arnica |
| 5-10% | hamamelis |
| 1-2% | calendula |
| 1-3% | glucosamine |
| 1-3% | salicylic acid |
| 1% | emu oil |
| 1% | wild yam |

FORMULA 2

| | |
|---|---|
| 60-70% | petrolatum/petroleum jelly |
| 10-15% | hamamelis |
| 10-15% | glucosamine |
| 1-3% | emu oil |
| 1-3% | salicylic acid |
| 1-2% | arnica |
| 1% | wild yam |
| 1-6% | calendula |

FORMULA 3

| | |
|---|---|
| 90% | petrolatum/petroleum jelly |
| 1-3% | hamamelis |
| 1-3% | glucosamine |
| 1-2% | salicylic acid |
| 1-2% | emu oil |
| 1% | arnica |
| 1% | wild yam |
| 1-4% | calendula |

All percentages in the above described formulations are by weight of the ingredients.

Experiment 1

Individuals with diagnosed bilateral osteoarthritis of the knees which were resistant to prior treatments including NSAIDs, cortisone injections and other traditional medical treatments including at least 6 weeks of physical therapy within the last year were given a 2 week period of treatment with the medicament of the present invention. Ages of the subjects were limited to 62-78. Each was administered a questionnaire which determined the level of present pain and difficulty with activities of daily life.

Pain scale 0=no pain
10=severe pain all the time

Activities of daily life 0=no restrictions
(AODL) 10=severe restrictions

Application of the medicament in a cream form was made twice a day, once in the morning and once at night. The cream was rubbed into the anterior and posterior aspect of the joint(s) involved.

Formula 1 Results

| | |
|---|---|
| Pre-Treatment | Post- Treatment |

| | Pain | AODL | Pain | AODL |
|---|---|---|---|---|
| Subject 1 | 9 | 8 | 4 | 1 |
| Subject 2 | 8 | 6 | 2 | 4 |
| Subject 3 | 8 | 8 | 4 | 2 |
| Subject 4 | 10 | 9 | 3 | 3 |

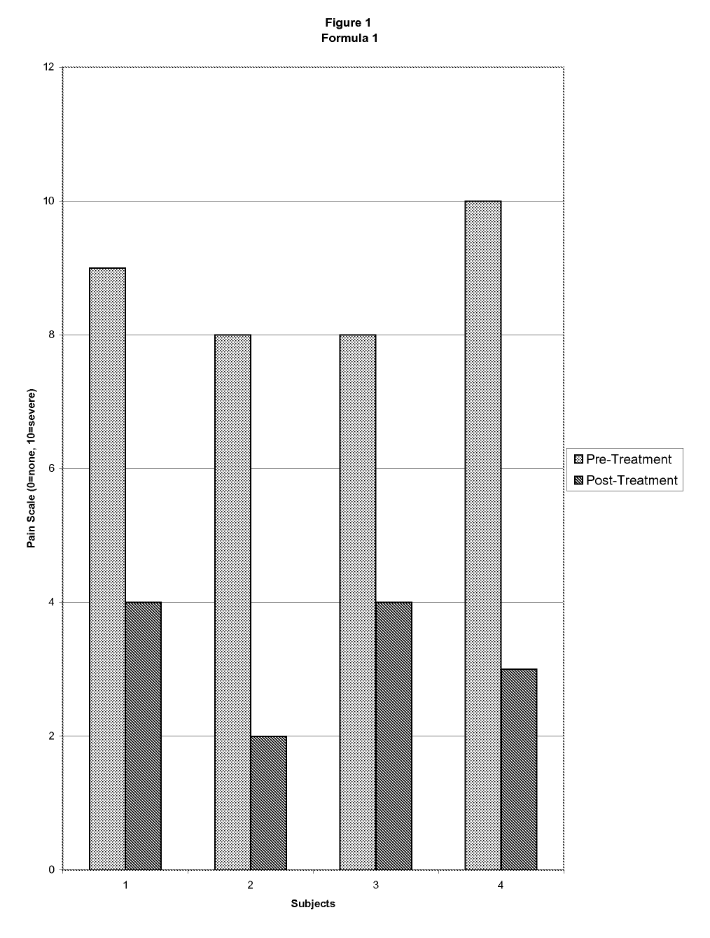

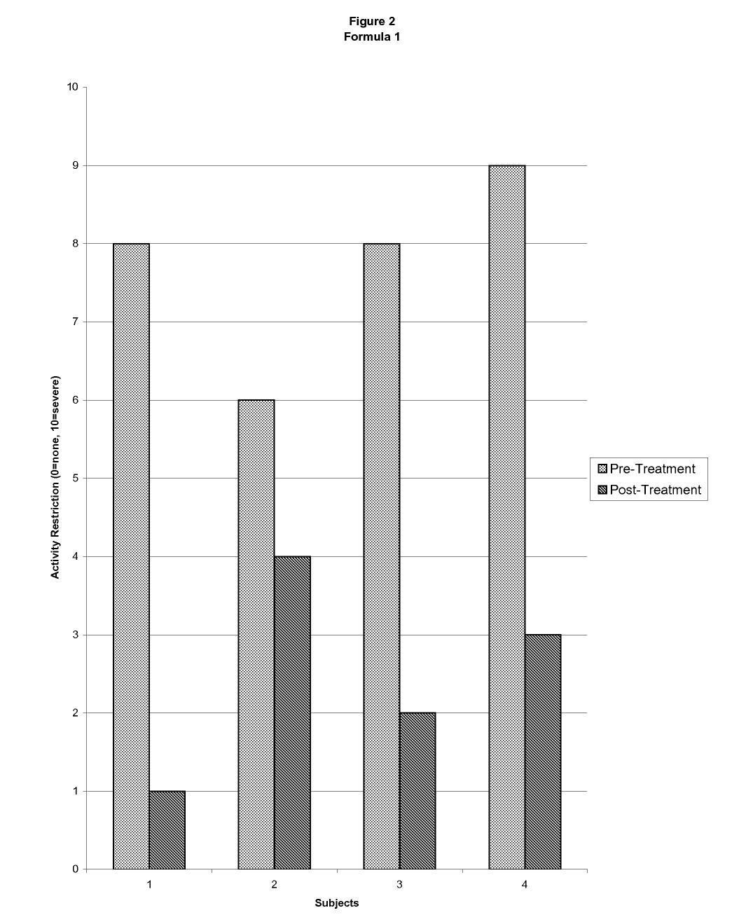

FIGS. 1 and 2 graphically demonstrates the changes in the pain scale and daily activity restrictions of the individuals treated with the medicament having the composition described in Formula 1. It can be seen that the subjects experienced a decrease in pain and less restrictions in their daily activities after treatment with the medicament of Formula 1.

Formula 2 Results

| | |
|---|---|
| Pre-Treatment | Post- Treatment |

| | Pain | AODL | Pain | AODL |
|---|---|---|---|---|
| Subject 1 | 7 | 8 | 5 | 6 |
| Subject 2 | 9 | 8 | 7 | 3 |
| Subject 3 | 8 | 8 | 6 | 4 |
| Subject 4 | 9 | 8 | 2 | 4 |
| Subject 5 | 6 | 5 | 2 | 2 |
| Subject 6 | 9 | 4 | 3 | 0 |

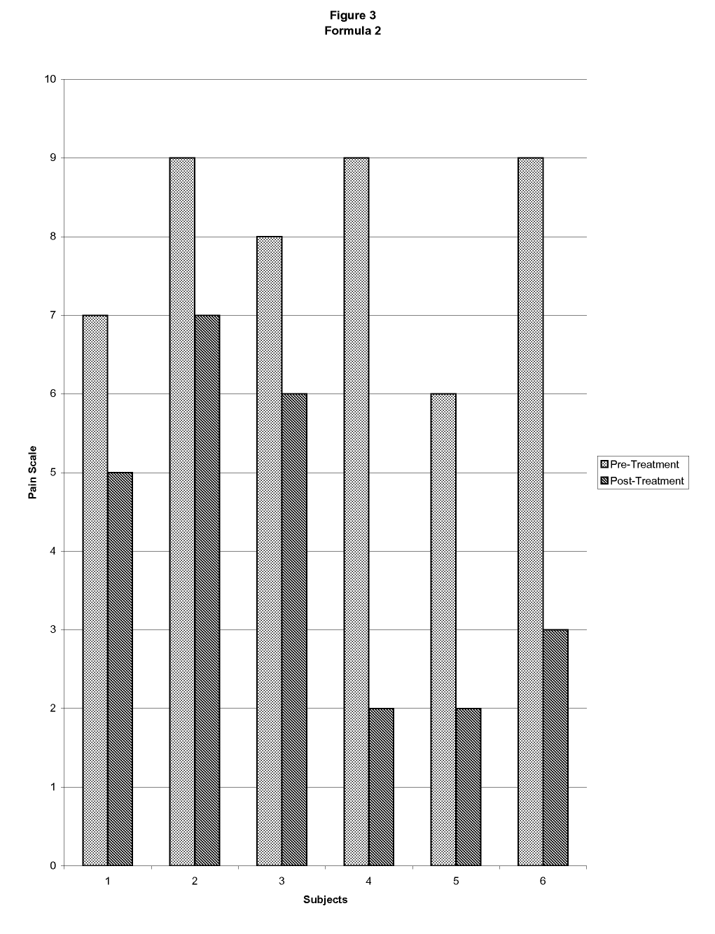

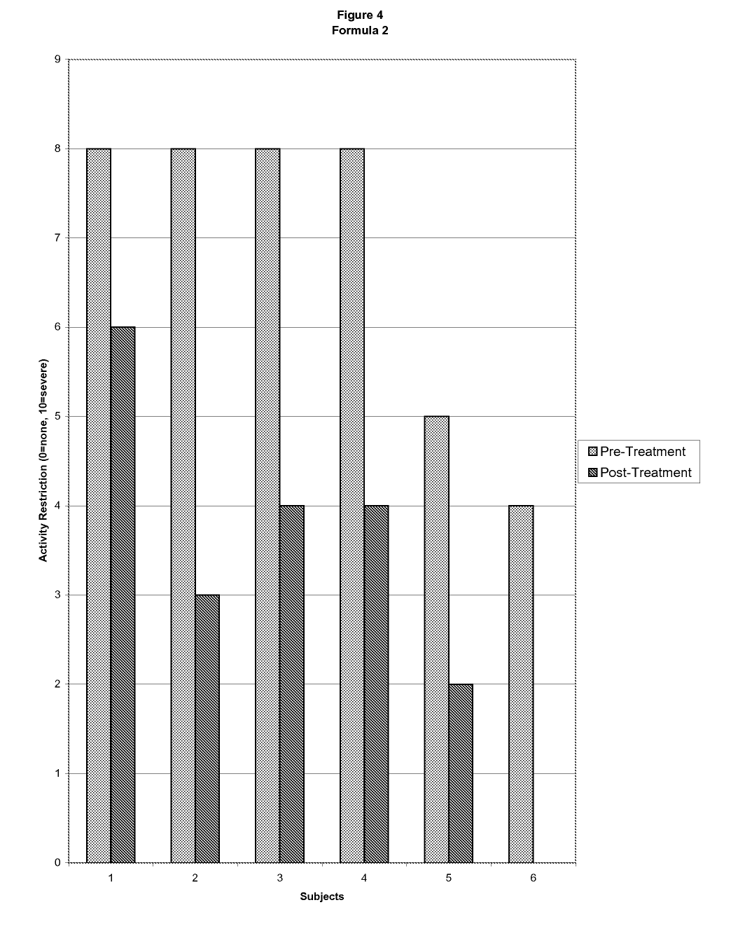

FIGS. 3 and 4 graphically demonstrates the changes in the pain scale and daily activity restrictions of the individuals treated with the medicament having the composition described in Formula 2. It can be seen that the subjects experienced a decrease in pain and less restrictions in their daily activities after treatment with the medicament of Formula 2.

Formula 3 Results

| | |
|---|---|
| Pre-Treatment | Post- Treatment |

| | Pain | AODL | Pain | AODL |
|---|---|---|---|---|
| Subject 1 | 7 | 9 | 1 | 3 |
| Subject 2 | 9 | 9 | 5 | 2 |
| Subject 3 | 10 | 9 | 6 | 3 |
| Subject 4 | 8 | 7 | 7 | 5 |
| Subject 5 | 8 | 5 | 1 | 2 |

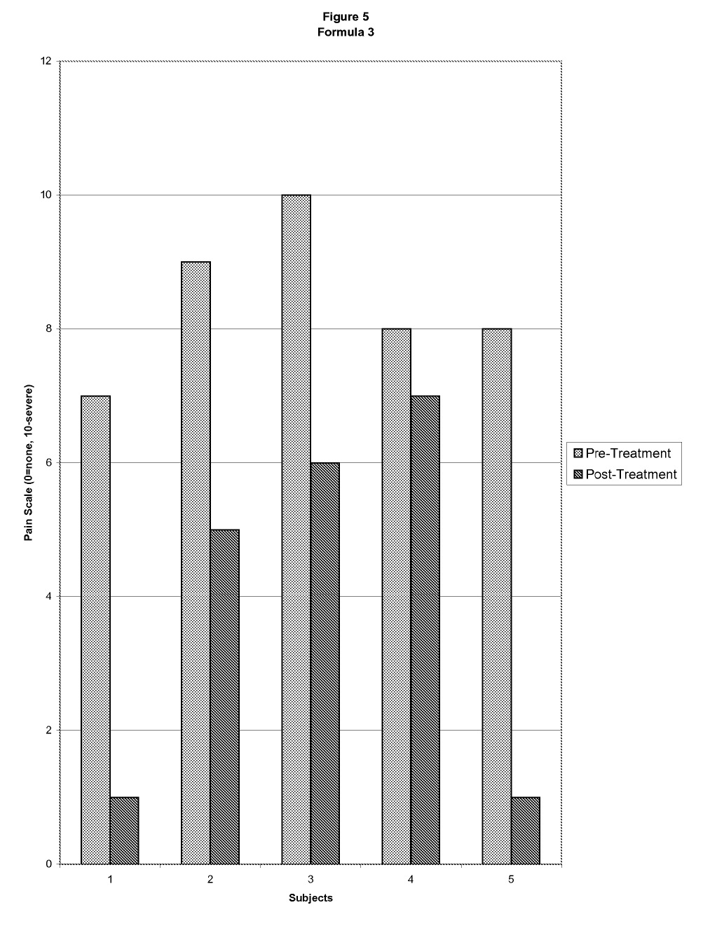

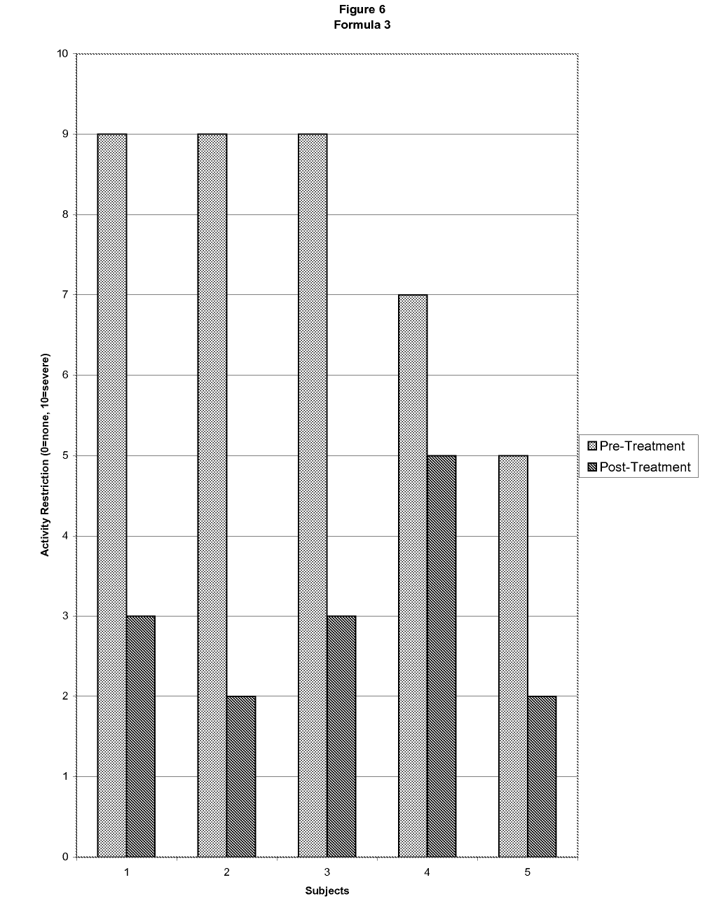

FIGS. 5 and 6 graphically demonstrates the changes in the pain scale and daily activity restrictions of the individuals treated with the medicament having the composition described in Formula 3. It can be seen that the subjects experienced a decrease in pain and less restrictions in their daily activities after treatment with the medicament of Formula 3.

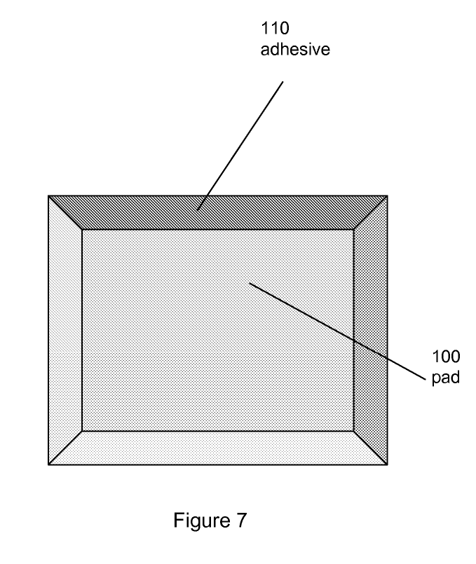

Each of the above formulations may be applied directly on the skin by hand, through the use of a self adhesive pad impregnated with the medicament, applied directly on the affected area and then the area is wrapped with a material sleeve or band, or on a pad impregnated with the medicament that is held in place by a sleeve or band. FIG. 7 illustrates a pad 100 with adhesive 110 around the edges for securing the pad to the affected area.

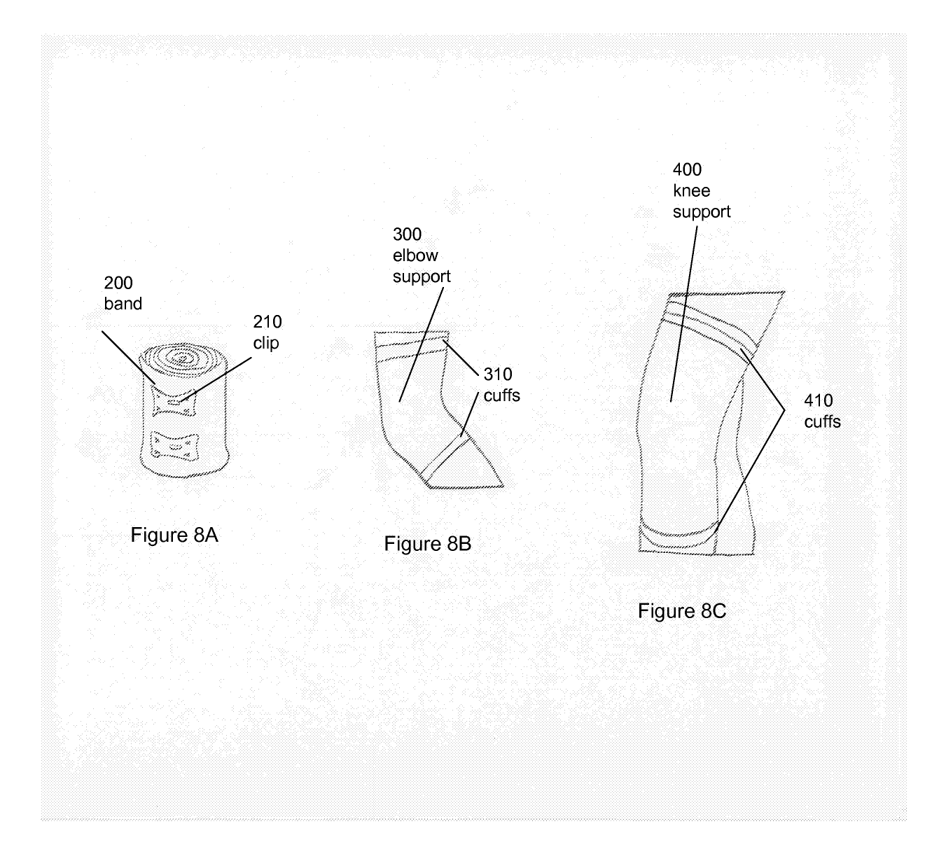

FIGS. 8A-C illustrate examples of a sleeve or band used to wrap directly around the affected area or to secure a pad containing the medicament in place on the affected area. The sleeve or band is made of cotton, wool or other material that augments penetration of the medicament into the skin. The sleeve or band should also be elastic in nature so as to provide a snug fit around the affected area. The sleeve or band may be secured in place using any of the pins, clips, laces, or hook and loop fasteners known to those in the art. FIG. 8A is a band 200, shown in the coiled position, that is wrapped around the affected area, then secured in places by clips 210. Alternatively, as seen in FIGS. 8B and 8C, the sleeve or band may a tubular omnidirectional woven product. FIG. 8B is sleeve 300 for use on the elbow. Sleeve 300 has a pair of cuffs 310 to assist in keeping sleeve 300 in place. FIG. 8C is a sleeve 400 for use on the knee. Sleeve 400 has a pair of cuffs 410 to assist in keeping sleeve 400 in place.

The carrier mechanism for the medicament may be in the form of an ointment, gel, cream, liquid solution, or any substance that is capable of being applied to the skin.

The foregoing description of the embodiments of the invention has been presented for the purposes of illustration and description. It is not intended to be exhaustive or to limit the invention to the precise form disclosed. Many modifications and variations are possible in light of this disclosure. It is intended that the scope of the invention be limited not by this detailed description, but rather by the claims appended hereto.

1. A medicament for the treatment of arthritis comprising a composition comprising arnica, calendula, glucosamine, salicylic acid, hamamelis, emu oil and wild yam.

2. The medicament of claim 1 wherein the composition further comprises petrolatum.

3. The medicament of claim 1 wherein the composition is in a range of approximately 70% to 85% by weight petrolatum, 5% to 10% by weight arnica, 5% to 10% by weight hamamelis, 1% to 2% calendula, 1% to 3% by weight glucosamine, 1% to 3% by weight salicylic acid, 1% by weight emu oil and 1% by weight wild yam.

4. The medicament of claim 1 wherein the composition is in a range of approximately 60% to 70% by weight petrolatum, 10% to 15% by weight hamamelis, 10% to 15% by weight glucosamine, 1% to 6% by weight calendula, 1% to 3% by weight salicylic acid, 1% to 3% by weight emu oil and 1% by weight wild yam.

5. The medicament of claim 1 wherein the composition is in a range of approximately 90% by weight petrolatum, 1% to 4% by weight calendula, 1% to 3% by weight hamamelis, 1% to 3% by weight glucosamine, 1% to 2% by weight salicylic acid, 1% to 2% by weight emu oil, 1% by weight arnica, and 1% by weight wild yam.

6. The medicament of claim 1 wherein the composition is formed into a substance suitable for topical administration selected from the group consisting of: an ointment, cream, gel, emulsion, liquid solution, or combination thereof.

7. The medicament of claim 1 wherein the composition is applied directly to an affected area.

8. The medicament of claim 1 wherein the composition is applied to an affected area twice a day.

9. The medicament of claim 1 wherein a pad is saturated with the composition and applied to an affected area, the pad having an adhesive for securing the pad to the affected area.

10. The medicament of claim 7 wherein the composition applied directly to the affected area is then wrapped with a sleeve.

11. The medicament of claim 9 wherein the pad saturated with the composition is then wrapped with a sleeve.

12. The medicament of claim 11 wherein the sleeve is made of material selected from the group consisting of: cotton, wool, and a combination thereof.

13. The medicament of claim 11 wherein the sleeve uses a fastening means selected from the group consisting of: pins, clips, laces, hook and loop fasteners, and a combination thereof.

14. The medicament of claim 11 wherein the sleeve is a tubular omnidirectional woven material.

15. A method for treating the physical symptoms of arthritis which comprises administering to an affected area an effective amount of a composition comprising amica, calendula, glucosamine, salicylic acid, hamamelis, emu oil and wild yam.

16. The method of claim 15 further comprising topically administering the composition to the affected area.

17. The method of claim 15 further comprising administering the composition to the affected area twice a day.

18. The method of claim 16 further comprising wrapping a sleeve around the affected area.

19. The method of claim 15 further comprising applying a pad saturated with the composition to the affected area.

20. The method of claim 19 further comprising wrapping a sleeve around the pad.

21. A medicament for the treatment of arthritis comprising a composition comprising ingredients selected from a group consisting of arnica, calendula, members of the Inula and Scorzonera genera, glucosamine, chondroitin sulfate, methylsulfonylmethane, salicylic acid, hamamelis, members of the Parrotiopsis, Fothergilla and Sycopsis genera, emu oil, ostrich oil, kiwi oil, cassowary oil, rhea oil, wild yam, members of the Viburnum, Apium, Althaea, and Filipendula genera, and a combination thereof.
